(19) United States
(12) Plant Patent Application Publication
(10) Pub. No.: US 2017/0127586 P1
(43) Pub. Date: May 4, 2017

(54) Citrus reticulata blanco plant named `Tamdori'

(50) ;

(71) Applicant: THE RURAL DEVELOPMENT ADMINISTRATION, REPUBLIC OF KOREA, CHUNGCHEONGNAM-DO (KR)

(72) Inventors: JAEHO PARK, SEOGWIPO-SI (KR); SANGWOOG KOH, SEOGWIPO-SI (KR); SUKMAN PARK, SEOGWIPO-SI (KR); MINJU KIM, SEOGWIPO-SI (KR); YONGHUN CHOI, SEOGWIPO-SI (KR); SU-HYUN YUN, SEOGWIPO-SI (KR); I-UNG YANG, SEOGWIPO-SI (KR)

(21) Appl. No.: 14/756,933

(22) Filed: Oct. 30, 2015

(51) Int. Cl. A01H 5/08

(52) U.S. Cl. PLT/201

(57) ABSTRACT

A new and distinct Citrus Reticulate Blanco plant named ‘Tamdori’, characterized by its upright plant type and strong vigor, fertile but self-incompatibility pollen, orange fruit color, germination stage in middle of April, blooming stage in late May, fruits start coloring in late October and finish coloring in late December, mature stage in middle of February, fruit weight at about 168.9 g, fruit skin thickness at about 2.3 mm and easy to peel, fruit flesh rate at about 79.9%.

Latin name of the genus and species of the plant claimed: Citrus reticulata Blanco.

Variety denomination: ‘Tamdori’.

### BACKGROUND OF THE INVENTION

The new and distinct cultivar is a product of a breeding program by the inventors by crossing a Japanese variety “Kiyomi’ and an American variety ‘Fortune” mandarin (as the pollen parent) in 2005. The seeds obtained by the cross were sowed in a greenhouse in the spring of 2006. The seedlings germinated from the seeds were upper-position grafted onto ‘satsuma’ mandarin in the spring of 2007. The plants started fruiting in 2011. In the same year, the first screening was carried out, and the one called Jegamna-40 was selected for good quality. In the last screening which carried out in 2013, Jegamna-40 was selected as an office upbringing new variety and named ‘Tamdori’.

After hybridization of the parents, the variety was planted by asexual propagation for three generations and there was no genetic variation, therefore, its traits are stable.

The fruit characteristics of Tamdori are in the table below. The test carried out from 2011 to 2013, in Citrus Research Station.

| Survey | Fruit | | | Thickness | Seed | | | Sugar |
|---|---|---|---|---|---|---|---|---|
| date | weight | Flesh | Fruit shape | of the skin | grain | Sugar | Acidity | acid |
| (yy.mm.dd) | (g) | rate(%) | index | (mm) | number | (° Bx) | (%) | ratio |
| 11.02.07 | 130.3 | 84.9 | 123 | 1.5 | 0.0 | 14.4 | 1.34 | 10.71 |
| 11.02.21 | 154.3 | 82.7 | 121 | 2.0 | 6.8 | 14.9 | 1.04 | 15.24 |
| 11.03.08 | 186.5 | 74.7 | 121 | 3.1 | 3.6 | 14.9 | 0.87 | 17.74 |
| 11.03.29 | 158.6 | 79.5 | 114 | 3.0 | 1.5 | 16.1 | 0.78 | 20.85 |
| 12.01.05 | 124.0 | 87.0 | 139 | 1.2 | 5.0 | 13.1 | 1.14 | 11.55 |
| 12.01.20 | 144.0 | 85.7 | 135 | 1.6 | 0.8 | 13.9 | 0.99 | 14.31 |
| 12.02.06 | 147.4 | 84.8 | 145 | 1.7 | 3.8 | 13.5 | 0.81 | 16.64 |
| 12.02.20 | 135.2 | 84.0 | 128 | 1.5 | 8.8 | 15.1 | 0.65 | 23.13 |
| 13.01.22 | 175.3 | 84.6 | 116 | 2.1 | 11.8 | 13.9 | 1.19 | 11.68 |
| 13.02.12 | 168.9 | 79.9 | 108 | 2.3 | 10.8 | 14.6 | 1.17 | 12.46 |
| 13.02.25 | 170.1 | 79.5 | 114 | 2.4 | 13.8 | 14.9 | 0.83 | 18.09 |

Fruit shape index=The transverse diameter÷The longitudinal diameter×100

### SUMMARY OF THE INVENTION

The cultivar ‘Tamdori’ has not been observed under every possible environmental conditions. Therefore, the may be variations in the phenotype due to varying environmental conditions such as temperature, day length and light intensity. However, the genotype will not vary.

The following traits have been repeatedly observed and are determined to be the characteristics of the new cultivar of Citrus reticulata Blanco. These attributes in combination distinguish ‘Tamdori’ as unique from and all other varieties of Citrus reticulata Blanco known to Inventors.

1. The plant type of ‘Tamdori’ is upright and the tree vigor is strong.

2. ‘Tamdori’ has fertile pollen but it is self-incompatibility.

3. The fruit color is orange.

4. Germination stage of ‘Tamdori’ is in the middle of April, and the blooming stage is in late May.

5. Fruits start coloring in late October and finish coloring in late December.

6. The mature stage is in the middle of February.

7. Fruit weight is about 168.9 g.

8. Fruit skin thickness is about 2.3 mm so it is easy to peel.

9. The fruit flesh rate is about 79.9%.

10. ‘Tandori’ is self-incompatibility, but when fertilized by other variety, several seeds can be gained.

11. Sugar of fruit juice is about 14.6° Bx, and the sugar acid ratio is about 12.46.

12. ‘Tamdori’ has small leaf, the leaf area is about 27.9 cm². The leaf length is about 9.9 cm, and the leaf width is about 4.3 cm.

13. The length of leaf petiole is about 19.2 mm and the thickness is about 1.0 mm.

Plants of the new cultivar ‘Tamdori’ are similar to plants of the seed parent ‘Kiyomi’ in most horticultural characteristics, however, the plants of the new cultivar ‘Tamdori’ produce fruit that are less acidic than the parent. Compared to the parent, ‘Tamdori’ produces fruit with skin thickness of 2.3 mm. Fruit of ‘Tamdori’ has a considerable number of seeds.

The new caltivar differs from the closest comparable plants as follows:

| Denom- | | | |
|---|---|---|---|
| ination | Characteristic in | | |
| of similar | which the similar | The expression of the characteristic | |
| variety | variety is different | Similar variety | Candidate variety |
| Kiyomi | Plant: type | medium | upright |
| | Plant: tree vigor | medium | strong |
| | Flower: the pollen | no pollen | has fertile pollen |
| | Fruit: weight | 291.6 g | 168.9 g |
| | Fruit: flesh rate | 72.6% | 79.9% |
| | Fruit: skin thickness | 4.6 mm | 2.3 mm |
| | Fruit: number of | 0.0 | 10.8 |
| | seeds | | |
| | Fruit juice: Sugar | 11.0 | 14.6 |
| | (° Bx) | | |
| | Fruit: sugar acid | 9.43 | 12.46 |
| | ratio | | |
| | Fruit: color | yellowish | medium |
| | | orange | orange |
| | Leaf: leaf area | about 72.8 cm² | about 27.9 cm² |
| | Leaf: leaf length | 15.5 cm | 9.9 cm |
| | Leaf: leaf width | 6.9 cm | 4.3 cm |
| | Leaf petiole: length | 23.1 mm | 19.2 mm |
| | Leaf petiole: | 1.7 mm | 1.0 mm |
| | thickness | | |
| | Mature stage | early (in the mid- | very early (in the |
| | | dle of March) | middle of February) |

### BRIEF DESCRIPTION OF THE DRAWINGS

The accompanying colored photographs illustrate the overall appearance and distinct characteristics of the new Citrus reticulata Blanco ‘Tamdori’ showing colors as true as is reasonably possible with colored reproduction of this type. These photos were taken in 2013 in Korea. Colors in the photographs may differ slightly from the color values cited in the detailed botanical description which accurately describes the color of the ‘Tamdori’.

FIG. 1 provides multiple views of typical fruits harvested from ‘Tamdori’. Top, bottom and side views of the whole, unpeeled fruit is shown as well as two cross-sectional views of the inside of the fruit.

FIG. 2 illustrates in full color typical foliage and fruits of ‘Tamdori’ on the tree.

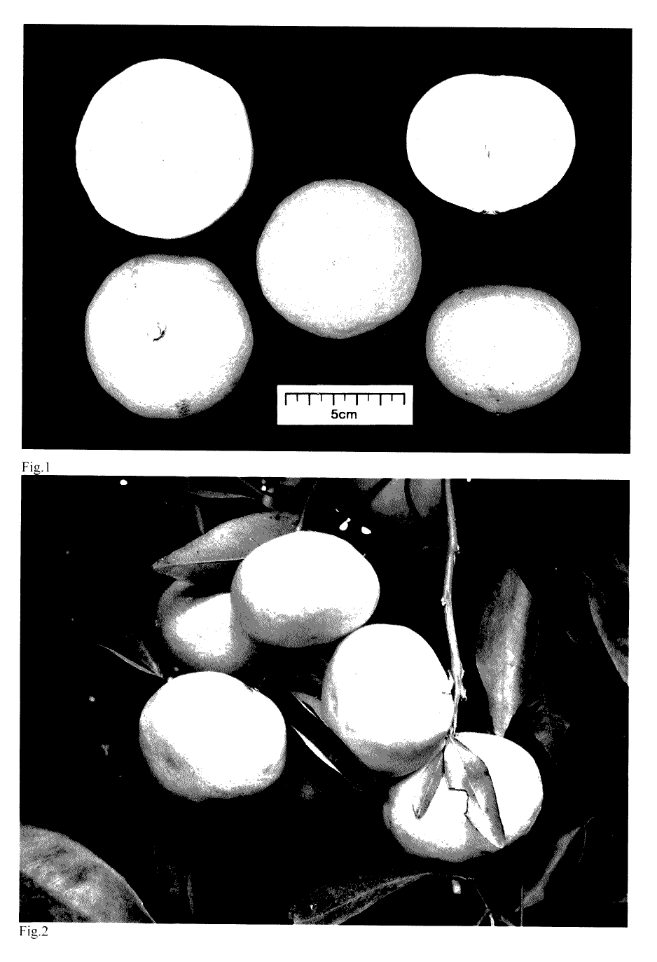

### DETAILED BOTANICAL DESCRIPTION

The following is a detailed description of the new cultivar ‘Tamdori’ plants as grown in Korea. The descriptors were taken between 2011 and 2013.
Botanical classification: Citrus reticulata Blanco ‘Tamdori’.
Propagation: ‘Tamdori’ is typically grafted onto rootstock of “Satsuma” mandarin.
Plant:
   Age of the plant described.—Approximately six years.
   Chromosome multiples.—2.
   Plant type.—Upright.
   Plant tree vigor.—Strong.
   Plant density of spines.—None or sparse.
Foliage:
   Leaf color.—Green.
   Leaf area.—27.9 cm² .
   Leaf length.—9.9 cm.
   Leaf width.—4.3 cm.
   Leaf length/width ratio.—2.29.
   Leaf petiole length.—23.1 mm.
   Leaf petiole thickness.—1.0 mm.
Flower:
   Flower calyx diameter.—3.6 mm.
   Flower petal length.—15.2 mm.
   Flower petal width.—6.1 mm.
   Flower petal length/width ratio.—2.5.
   Flower stamen length.—8.0 mm.
   Flower anther color.—Light yellow.
   Flower activity pollen.—Present.
Fruit:
   Fruit weight.—168.9 g.
   Fruit length.—65.6 mm.
   Fruit width.—70.7 mm.
   Fruit length/width ratio.—0.93.
   Fruit neck.—Absent.
   Fruit areola.—Absent.
   Fruit surface color.—Medium orange.
   Fruit surface roughness.—Rough.
   Fruit fresh color.—Light orange.
   Fruit skin thickness.—2.3 mm.
   Fruit skin strength.—Medium.
   Fruit flesh rate.—79.9%.
   Fruit number of seeds.—10.8.
   Fruit sugar acid ratio.—12.46.
   Fruit juice total soluble solids.—14.6° Bx.
   Fruit juice acidity.—1.17.
   Mature stage.—Very early.
   Parthenocarpy.—Present.
   Self-incompatibility.—Present.
   Tree vigor.—Strong.
   Other fruit features.—For the very early mature stage, ‘Tamdori’ should be protected cultured. Pruning on ‘Tamdori is the same as other variety which belongs to Citrus reticulata Blanco.

What is claimed is:

1. A new and distinct cultivar of Citrus reticulata Blanco plant named “Tamdori” as herein illustrated and described.
